(19) United States
(12) Patent Application Publication
(10) Pub. No.: US 2007/0009601 A1
(43) Pub. Date: Jan. 11, 2007

(54) Composition and method for the treatment of water related ear disorders

(75) Inventor: Peter Tunguy-Desmarais, Kwazulu-Natal. (ZA)

Correspondence Address: BIRCH STEWART KOLASCH & BIRCH, PO BOX 747, FALLS CHURCH, VA, 22040-0747, US

(73) Assignee: Swimseal International (Pty) Ltd., Gauteng (ZA)

(21) Appl. No.: 10/562,639

(22) PCT Filed: Jul. 2, 2004
(86) PCT No.: PCT/ZA04/00074

§ 371 Date: May 16, 2006

(30) Foreign Application Priority Data
Jul. 2, 2003 (ZA) 03/5160

(51) Int. Cl. A61K 36/61; A61K 9/14

(52) U.S. Cl. 424/486; 424/769

(57) ABSTRACT

This invention provides a method for the treatment of water related outer ear canal disorders comprising the steps of applying a water resistant and antimicrobial film topically to the external ear canal. The invention includes a composition for the treatment of water related outer ear canal disorders comprising a water repellent agent in the form of a polysiloxane and an antimicrobial agent in the form of the essential oil of Melaleuca alternifolia or Tea Tree oil in which the two oils are combined to produce a film forming liquid that is capable of being applied topically to the external ear canal by instillation in droplet form.

### BACKGROUND TO THE INVENTION

This invention relates to a composition and method for the treatment of water related ear canal disorders.

### SUMMARY OF THE INVENTION

According to this invention a method for the treatment of water related outer ear canal disorders comprises the steps of applying a water resistant and antimicrobial film topically to the external ear canal.

The invention includes a composition for the treatment of water related outer ear canal disorders comprising a water repellent agent and an antimicrobial agent in a film forming liquid capable of being instilled in the outer ear canal in droplet form.

The water repellent agent may be constituted by a water repellent silicone polymer adapted, in combination with the antimicrobial agent or on its own, to constitute a film forming liquid.

The silicone polymer is preferably constituted by a polysiloxane, a group of silicone oils which are biologically inert, water repellent, film forming liquids.

The antimicrobial agent is preferably a natural product capable of acting against a broad range of bacterial and fungal infections, the preferred antimicrobial agent being Tea Tree oil, the essential oil of the Tea Tree (Melaleuca alternifolia), which is a natural antiseptic.

In the preferred form of the invention, therefore, the composition comprises a water repellent agent in the form of a polysiloxane and an antimicrobial agent in the form of the essential oil of Melaleuca alternifolia or Tea Tree oil.

The two oils combine to produce a film forming liquid that is capable of being applied topically to the external ear canal by instillation in droplet form.

### DESCRIPTION OF EMBODIMENTS OF THE INVENTION

The physiology of the skin lining the external ear canal is well known to otological clinicians, but for some reason, the application of a physiological basis to the pharmacological treatment of water related outer ear canal disorders seems to have been neglected.

A normal, healthy human outer ear canal is lined with cerumen glands that secrete a waxy exudate known as cerumen which aids in trapping airborne debris, repelling water and acidifying the epithelial surface of the canal. This assists in minimising bacterial and fungal infections within the canal. Fine hairs within the outer ear canal impel the ear wax gradually outwardly, carrying any dirt it has trapped. Eventually, the ear wax migrates out of the outer ear canal, dries and falls out of the ear in small, unnoticeable flakes.

Inflammation of the outer ear canal often arises as a result of improper ear care or when too much water gets in the ear. The warm, humid environment of the outer ear canal and the removal of protective ear wax during swimming, showering or over-enthusiastic cleaning combine to provide ideal conditions for the proliferation of harmful bacteria and fungi. In addition, exposure to large amounts of water such as, for instance, during swimming, results in the epithelial lining of the outer ear canal becoming more alkaline, leading to an increased growth in bacteria, fungal and viral organisms which capitalise on the more alkaline conditions.

The remedies conventionally prescribed for avoiding the ingress of water into the outer ear canal include the use of ear plugs of various types to prevent the ingress of water or the avoidance of water altogether.

An alternative to ear plugs is to apply petroleum jelly to the ear canal as a means of plugging the canal. This often results in the formation of a matrix with desquamating skin within the canal, further encouraging water retention in the ear canal.

The remedies conventionally prescribed for removing excess water from the outer ear canal include the use of hairdryers to dry the ear after the ingress of water or instilling an alcohol containing drop to disperse water. This is the most favoured approach.

Both these remedies have their place in the treatment of water related outer ear canal disorders, but it is generally accepted that the use of alcohol drops in particular has a serious shortcoming. Whilst assisting in water dispersion and drying of the skin of the outer ear canal, alcohol tends to cause excessive stripping of cerumen which leaves the skin excessively dry and subject to the effects of humidity and water. Upon further exposure to moisture, the skin becomes macerated and soggy, and encourages the growth of bacteria and fungi.

In contrast, the method and composition of this invention propose the use of the combination of a water repellent agent and an antimicrobial agent in a film forming liquid capable of being instilled in the outer ear canal in droplet form.

The water repellent agent is a water repellent silicone polymer, preferably a polysiloxane, a group of silicone oils which are biologically inert, water repellent film forming liquids.

Polysiloxane or silicone oil is capable, on its own and in combination with the antimicrobial agent, to constitute a film forming liquid.

The antimicrobial agent is a natural product capable of acting against a broad range of bacterial and fungal infections, the preferred antimicrobial agent being Tea Tree oil, the essential oil of the Tea Tree (Melaleuca alternifolia), which is a natural antiseptic.

The two oils combine to produce a film forming liquid that is capable of being applied topically to the external ear canal by instillation in droplet form.

The composition may conveniently comprise between 75% and 98%, by volume of polysiloxane and between 2% and 25% by volume of Tea Tree oil.

The composition of this invention is adapted to seal the skin of the outer ear canal with a water repelling layer of silicone polymer whilst bringing to bear the natural antimicrobial action of Tea Tree oil.

The silicone polymer is inert and exerts its action purely because of its physical properties. It is preferably used in the composition as relatively thin, non-viscous oil. In this form, it has good film-forming properties on human skin. Instead of the physically invasive use of ear plugs or the equally ineffectual use of petroleum jelly, the silicone polymer, forms a thin, chemically inert, water repellent film on the skin of the outer ear canal.

Tea tree (Melaleuca alternifolia) and particularly its essential oil are widely regarded as some of the most important natural antibacterial and antiseptic treatments. Tea Tree oil is used for treating stings, burns, wounds and skin infections of all kinds. The essential oil, which is obtained from the leaves and twigs of the plant, is strongly antiseptic, diaphoretic and expectorant. It stimulates the immune system and is effective against a broad range of bacterial and fungal infections. The essential oil of Melaleuca alternifolia is non-irritant and it is used externally in the treatment of a number of skin disorders, for which uses it is normally diluted with a carrier oil such as almond oil.

In the composition of the invention, the Tea Tree oil is used as the essential oil of Melaleuca alternifolia, diluted with polysiloxane.

In use, a person hoping to prevent water related ear problems will instill a few drops of the composition in both ears a short while before entering water. A person who regularly experiences such ear problems could also use the composition more regularly, between two and four times a week, for instance, as a preventative measure. Being largely natural and devoid of any irritants, preservatives, alcohol or other pharmacologically active substances, the composition will be well tolerated in such a preventative treatment regime.

1. A method for the treatment of water related outer ear canal disorders comprising the steps of applying a water resistant and antimicrobial film topically to the external ear canal.

2. A composition for the treatment of water related outer ear canal disorders comprising a water repellent agent and an antimicrobial agent in a film forming liquid capable of being instilled in the outer ear canal in droplet form.

3. A composition according to claim 2 in which the water repellent agent is constituted by a water repellent silicone polymer adapted, in combination with the antimicrobial agent or on its own, to constitute a film forming liquid.

4. A composition according to claim 3 in which the silicone polymer is constituted by a polysiloxane.

5. A composition according to claim 2 in which the antimicrobial agent is a natural product capable of acting against a broad range of bacterial and fungal infections.

6. A composition according to claim 5 in which the antimicrobial agent is Tea Tree oil (the essential oil of the Tea Tree (Melaleuca alternifolia)).

7. A composition for the treatment of water related outer ear canal disorders comprising a water repellent agent in the form of a polysiloxane and an antimicrobial agent in the form of the essential oil of Melaleuca alternifolia or Tea Tree oil in which the two oils are combined to produce a film forming liquid that is capable of being applied topically to the external ear canal by instillation in droplet form.

8. A composition according to claim 3 in which the antimicrobial agent is a natural product capable of acting against a broad range of bacterial and fungal infections.

9. A composition according to claim 4 in which the antimicrobial agent is a natural product capable of acting against a broad range of bacterial and fungal infections.
